(19) United States
(12) Patent Application Publication
(10) Pub. No.: US 2005/0090506 A1
(43) Pub. Date: Apr. 28, 2005

(54) Insecticidal perfluoroalkylthiazole derivatives

(75) Inventors: Isao Iwataki, Gainesville, FL (US); Anatoliy Vakulenko, Gainesville, FL (US); Rie Sakamoto, Kanagawa (JP); Makio Yano, Kanagawa (JP)

Correspondence Address: DENNIS G. LAPOINTE;LAPOINTE LAW GROUP, PL, PO BOX 1294, TARPON SPRINGS, FL, 34688-1294, US

(73) Assignee: Nippon Soda Co., Ltd.

(21) Appl. No.: 10/728,043

(22) Filed: Dec. 4, 2003

Related U.S. Application Data
(63) Continuation-in-part of application No. 10/692,993, filed on Oct. 24, 2003, now abandoned.

(51) Int. Cl. A61K 31/44; A01N 43/40; A01N 43/78

(52) U.S. Cl. 514/256; 514/370; 514/342

(57) ABSTRACT

Novel 4-perfluoroalkylthiazole derivatives and the use as an insecticide and acaricide of the compounds of formula (1): 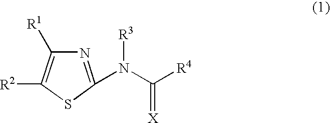 wherein
R¹ is C₂F₅, n-C₃F₇, i-C₃F₇, R² is H, halogen, cyano, alkoxycarbonyl, hydroxymethyl, haloalkyl, alkylthioalkyl, alkoxyalkyl, acyloxyalkyl, formyl, thiocyanatoalkyl, alkoxyiminomethyl, benzyloxyiminomethyl being optionally substituted by one or more of substituents, styryl being optionally substituted by one or more of substituents or phenyl being optionally substituted by one or more of substituents, R³ is H, alkyl, alkoxyalkyl or alkoxy, R⁴ is aryl, groups, being optionally substituted by one or more of substituents, X is O, S or NR⁵, R⁵ is alkyl, cycloalkyl, alkoxy, alkenylalkyloxy or alkynylalkyloxy.

### RELATED APPLICATION

This application is a continuation-in-part of U.S. patent application Ser. No. 10/692,993, filed Oct. 24, 2003.

### BACKGROUND OF THE INVENTION

1. Field of the Invention

The present invention relates to novel thiazole derivatives, which have insecticidal and acaricidal activity. The preparation and use, in agriculture and horticulture, of agrochemical compositions containing these novel insecticidal thiazoles are also disclosed.

2. Description of the Related Art

It is known in the art that certain 4-pentafluoroethylthiazole derivatives such as those disclosed in Japan Kokai Koho Heisei 8-311026, WO-9522523 have herbicidal activity and in EP-480902 have herbicide antidote properties. The present invention concerns the novel 4-perfluoroalkylthiazole derivatives which have excellent insecticidal and acaricidal activity.

### SUMMARY OF THE INVENTION

In accordance with the present invention, thiazole derivatives are provided having the formula (1): 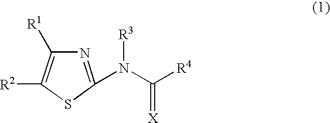
wherein
R¹ is C₂F₅, n-C₃F₇, i-C₃F₇,
R² is H, halogen, cyano, alkoxycarbonyl, hydroxymethyl, haloalkyl, alkylthioalkyl, alkoxyalkyl, acyloxyalkyl, formyl, thiocyanatoalkyl, alkylsulfonyl, alkylthio, alkoxyiminomethyl, benzyloxyiminomethyl being optionally substituted by one or more of halogen, alkyl, alkoxy, cyano, nitro, vinyl being optionally substituted by one or more halogen, nitro or cyano, styryl being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy; or phenyl being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy; thiocyanatoalkyl,
R³ is H, alkyl, alkoxyalkyl or alkoxy,
R⁴ is aryl, groups, being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkylsulfonyl, haloalkenyl, alkoxycarbonylalkyl or alkoxycarbonylalkoxy,
X is O, S or NR⁵,
R⁵ is alkyl, cycloalkyl, alkoxy, alkenylalkyloxy or alkynylalkyloxy.

The present invention is directed to agrochemical compositions comprising as an active ingredient at least one of the novel thiazole derivatives of the present invention, as well as to the use of these active ingredients or compositions for pest control, and, in particular as insecticides and acaricides useful in agriculture and horticulture.

For a better understanding of the present invention, reference is made to the following description and its scope will be pointed out in the appended claims.

### DETAILED DESCRIPTION OF THE INVENTION

For purposes of the present invention the general terms used hereinabove and hereinbelow have the following meanings, unless otherwise defined:

Alkyl groups are, in accordance with the number of carbon atoms, straight-chain or branched and will typically be methyl, ethyl, n-propyl, isopropyl, n-butyl, sec-butyl, isobutyl, tert-butyl, n-amyl, tert-amyl, 1-hexyl, or 3-hexyl.

Cycloalkyl groups are generally cyclopropyl, cyclobutyl, cyclopentyl, cyclohexyl.

Halogen and halo substituents will be understood generally as meaning fluoro, chloro, bromo, iodo. chloro, bromo, or iodo are preferred meanings in this invention.

Haloalkyl can contain identical or different halogenatoms, typically fluoromethyl, difluoromethyl, difluorochloromethyl, trifluoromethyl, chloromethyl, trichloromethyl.

Fluoroalkyl is generally fluoromethyl, difluoromethyl, trifluoromethyl, pentafluoroethyl, n-heptafluoroprpyl, n-nonafluorobutyl, n-undecafluoropentyl, n-tridecafluorohexyl and preferably trifluoromethyl and pentafluoroethyl.

Alkoxy is typically methoxy, ethoxy, propyloxy, isopropyloxy, n-butyloxy, isobutyloxy, sec-butyloxy, and tert-butyloxy. Methoxy and ethoxy are preferred.

Aryl is typically substituted phenyl or naphthyl, furyl, thienyl, six-membered heteroaromatic ring system such as pyridinyl, pyrimidinyl, pyridazinyl, pyrazinyl, triazinyl (1,2,3-, 1,2,4- and 1,3,5-), quinolinyl, isoquinolinyl, cinnolinyl, quinazolinyl, quinoxalinyl, indolinyl, isoindolinyl, five membered heteroaromatic ring such as thiazolyl, isothiazolyl, oxazolyl, isoxazolyl, pyrazolyl, thiadiazolyl oxadiazolyl, pyrollyl, imidazolyl, triazolyl (1,2,3- and 1,2,4-), tetrazolyl, fused five membered rings such as benzofuranyl, benzothienyl, benzimidazolinyl; being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy.

Alkenyl and alkynyl groups preferably contain from 2 to 6, more preferably from 2 to 4, carbon atoms. They can be in the form of straight or branched chains, and, where appropriate, the alkenyl groups can be of either (E)- or (Z)-configuration. Examples are vinyl, allyl, propargyl.

The alkylenedioxy groups are optionally substituted with halogene (especially fluorine) and are such as methylenedioxy or difluoromethylenedioxy. Alkoxyalkyl is C₁-C₄ alkoxy(C₁-C₄)alkyl such as methoxymethyl or ethoxymethyl.

The present invention provides the use as insecticides or acaricides of thiazole derivatives having the following formula (1): 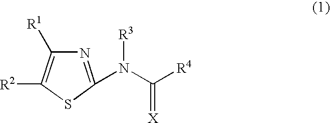
wherein R¹ is C₂F₅, n-C₃F₇, i-C₃F₇,
R² is H, halogen, cyano, alkoxycarbonyl, hydroxymethyl, haloalkyl, alkylthioalkyl, alkoxyalkyl, acyloxyalkyl, formyl, thiocyanatoalkyl, alkylsulfonyl, alkylthio, alkoxyiminomethyl, benzyloxyiminomethyl being optionally substituted by one or more of halogen, alkyl, alkoxy, cyano, nitro, vinyl being optionally substituted by one or more halogen, nitro or cyano, styryl being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy, or phenyl being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy; thiocyanatoalkyl,
R³ is H, alkyl, alkoxyalkyl or alkoxy,
R⁴ is aryl (especially phenyl, naphthyl, pyridinyl, pyrimidinyl, thienyl, furyl, thiazolyl, isothiazolyl, pyrazolyl, oxazolyl, isoxazolyl, thiadiazolyl (1,2,4, and 1,3,4), oxadiazolyl (1,2,4- and 1,3,4); being optionally substituted by one or more of halogen, cyano, alkyl, haloalkyl, alkoxy, nitro, alkoxycarbonyl, alkylcarbonyloxy, alkylcarbonyl, amino, alkylamino, haloalkoxy, alkylthio, or alkylsulfonyl, haloalkylsulfonyl), X is O, S or NR⁵ R⁵ is alkyl, cycloalkyl, alkoxy, alkenylalkyloxy or alkynylalkyloxy.

Examples of specific compounds of formula (1), which are of use as insecticides and acaricides include the compounds listed in Table 1.

A compound of formula (1) wherein R² is halogen and X is oxygen can be prepared by reacting a compound of formula (2): 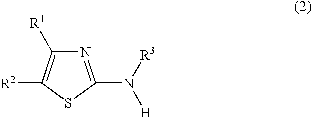
with a compound R⁴C(O)Cl in the presence of an organic base such as pyridine and triethylamine, or inorganic base such as sodium carbonate, potassium carbonate, sodium hydroxide and sodium hydride.

A compound of formula (1) wherein R² is halogen and X is S can be prepared by reacting a compound of formula (1) wherein X is oxygen with phosphorous pentasulfide in a high boiling solvent such as pyridine, picoline, lutidine, or xylene, sulfolane or via the corresponding imidoyl chlorides.

A compound of formula (1) wherein R² is aryl, styryl, haloalkyl, hydroxymethyl, alkoxyalkyl, alkylthioalkyl, formyl, cyano, substituted vinyl may prepared via the corresponding 5-alkoxycarbonyl derivatives.

Surprisingly, it has now been found that the novel compounds of formula (1) have, for practical purposes, a very advantageous spectrum of activities for protecting plants against insect and acarine pests, include such as Coleoptera, Diabrotica, Diptera, Homoptera and Lepidoptera, Heteroptera, Thysanoptera, Orthoptera and Acarina. The pests include those pests associated with agriculture, horticulture and animal husbandry, forestry, the storage of products of vegetable origin, such as fruit, grain, and timber, and also those pests associated with the transmission of diseases of man and animals. Examples of insect and acarine pest species which may be controlled by the compounds of formula (1) include: Pieris brassicae (white butterfly), Pseudaletia separata (rice armyworm), Heliothis virescens (tobacco budworm), Trialeurodes spp. (white flies), Aedes aegypti (mosquito), Agrotis spp. (cutworms), Blatta orientalis (cockroach), Anopheles spp. (mosquitos), Chilo partellus (maize stem borer), Culex spp. ((mosquitos), Dysdercus fasciatus (capsid), Musca domestica (housefly), Plutella xylostella (diamond back moth), Aonidiella spp. (scale insects), Bemisia tabaci (sweetpotato white fly), Blattella germanica (German cockroach), Myzus persicae (green peach aphid), Aphis gossypii (cotton aphid), Aphis fabae (bean aphid), Periplaneta americana (American cockroach), Phaedon cochleariae (mustard beetle), Spodoptera littoralis (cotton leafworm), Chortiocetes terminifera (locust), Diabrotica spp. (rootworms), Nilaparvata lugens (brown rice planthopper), Nephotettix cincticeps (green rice leafhopper), Tetranychus cinnabarinus (carmine spider mite), Panonychus ulmi (European red mite), Phyllocoptruta oleivora (citrus rust mite), Panonychus citri (citrus red mite), Tetranychus urticae (two-spotted spider mite), Polyphagotarsonemus latus (brode mite) and Brevipalpus spp. (mites).

Compounds of the formula (1) are nomally used in the form of compositions and can be applied to the crop and/or plant to be treated, simultaneously with or in succession with other compounds such as fertilizers, micronutrient donors or other preparations which influence the growth of plants. The thiazole derivatives of formula (1) can also be selectively combined with herbicides, as well as, other insecticides, fungicides, bactericides, nematocides, molluscicides or mixtures of several of these preparations and, if desired together with further carriers, surfactants or application promoting adjuvants employed in the art of formulation. In some cases, by mixing of the thiazole derivatives of formula (1) with other insecticides results synergistic insecticidal activity. When applying the compound of the present invention in a practical way, the compound may be applied in a form as it is without adding other components. When the compound of the present invention is applied for plant protection purpose, the compound can be prepared into general types of formulations for plant protection use, such as wettable powder, granules, dust, emulsifiable concentrate, water soluble powder, suspension concentrate, flowable liquid, and so on.

In case the compound of the present invention is prepared into a solid type formulation, appropriate additives and carriers may be incorporated with the compound. Examples of the additive and the carrier include phytogenic powders, such as soybean powder and flour, mineral fine powders, such as diatomaceous earth, apatite, gypsum, talc, bentonite, pyrophyllite and clay, and organic and inorganic compounds, such as sodium benzoate, urea and Glauber's salt. In case the compound of the present invention is prepared into a liquid type formulation, an appropriate solvent is used for dissolving or dispersing the compound in the liquid type formulation. Examples of the solvent used for the liquid formulation include petroleum fractions, such as kerosine, xylene and solvent naphtha, cyclohexane, cyclohexanone, dimethylformamide, dimethylsulfoxide, alcohol, acetone, methyl isobutyl ketone, mineral oils, vegetable oils and water.

In addition, in order to provide uniformity and stability to the compound in the prepared formulations, it is possible to add surface active agents into each formulation upon necessity. There is no limitation for the surface active agent, and examples of the surface active agent that can be added to the above-mentioned formulations include nonionic surface active agents, such as polyoxyethylene-added alkyl ether, polyoxyethylene-added higher fatty acid ester, polyoxyethylene-added sorbitan higher fatty acid ester and polyoxyethylene-added tristyryl phenyl ether, a sulfate ester of polyoxyethylene-added alkyl phenyl ether, an alkyl benzene sulfonate, a polycarbonate, a lignin sulfonate, a formaldehyde condensate of alkyl naphthalene sulfonate, and a copolymer of isobutylene and maleic anhydride.

In general, the content of an active ingredient in each of the formulations recited above is preferably in a range of from 0.01 to 90% by weight, and more preferably from 0.05 to 85% by weight based on the total weight of the formulation. Each of the prepared formulations, such as wettable powder, emulsifiable concentrate, suspension concentrate and flowable solution, is diluted with water to be prepared and adjusted into the suspension or emulsion with a desired concentration, and is applied to crop plants. For the formulations, such as granular and dust formulations, the formulation itself is directly applied to the target crop plants or soil.

Needless to say that the compound alone according to the present invention has sufficient insecticidal and acaricidal activity, however, it can be combined for the use with one or more of various types of other plant protection chemicals, for example, fungicides, insecticides, acaricides and synergists.

Hereunder, representative examples for fungicides, insecticides, acaricides and plant growth regulators those which can be combined to use with the compound according to the present invention will be recited below.

Fungicides:

Captan, Folpet, Thiuram, Ziram, Zineb, Maneb, Mancozeb, Propineb, Polycarbamate, Chlorothalonil, Quintozene, Captafol, Iprodione, Procymidone, Vinclozolin, Fluorimide, Cymoxanil, Mepronil, Flutolanil, Pencycuron, Oxycarboxine, Fosetyl aluminium, Propamocarb, Triadimefon, Triadimenol, Propiconazole, Diclobutrazol, Bitertanol, Hexaconazol, Microbutanil, Flusilazole, Etaconazole, Fluotrimazole, Flutriafen, Penconazole, Diniconazole, Cyproconazole, Fenarimol, Triflumizole, Prochloraz, Imazalyl, Pefurazoate, Tridemorph, Fenpropimorph, Triforine, Buthiobate, Pyrifenox, Anilazine, Polyoxins, Metalaxyl, Oxadixyl, Furalaxyl, Isoprothiolane, Probenazole, Pyrrolenitrine, Blastocidin-S, Kasugamycin, Balidamycin, Dihydrostreptomycin sulfate, Benomyl, Carbendazim, Thiophanate methyl, Hymexazol, Basic copper chloride, Basic copper sulfate, Fentin acetate, Triphenyltin hydroxide, Diethofencarb, Metasulfocarb, Quinomethionate, Binapacryl, Lecithin, Sodium hydrogencarbonate, Dithianone, Dinocap, Fenaminosulf, Diclomezine, GuazatineDodine, IBP, Edifenphos, Mepanipyrim, Ferimzone, Trichlamide, Metasulfocarb, Fluazinam, Ethoquinolac, Dimetomorph, Pyroquilon, Tecloftalam, Fthalide, Fenazine oxide, Thiabedazole, Tricyclazole, Vinclozolin, Cymoxanil, Cyclobutanil, Guaztine, Propamocarb hydrochloride, Oxolinic acid.

Insecticides and Acaricides:

Organophosphorous and carbamate insecticides: Fenthion, Fenitrothion, Diazinon, Chlorpyrifos, ESP, Vamidothion, Fenthoate, Dimethoate, Formothion, Malathion, Trichlorfon, Thiometon, Phosmet, Dichlorvos, Acephate, EPBP, Methyl parathion, Oxadimeton methyl, Ethion, Salithion, Cyanophos, Isoxathione, Pyridafenthion, Phosalone, Methidathion, Sulprofos, Chlorfevinphos, Tetrachlorvinphos, Dimethylvinphos, Propaphos, Isofenphos, Ethyl thiometon, Profenophos, Pyraclofos, Monocrotophos, Azinphos methyl, Aldicarb, Methomyl, Dithiocarb, Carbofuran, Carbosulfan, Benfuracarb, Furathiocarb, Propoxur, BPMC, MTMC, MIPC, carbaryl, Pyrimicarb, Ethiofencarb, Fenoxycarb, cartap, thiocyclam, bensultap, etc.
Pyrethroid insecticides: Permethrin, Cypermethrin, Deltamethrin, Fenvalerate, Fenpropathrin, Pyrethrin, Allethrin, Tetramethrin, Resmethrin, Dimethrin, Propathrin, Fenothrin, Prothrin, Fluvalinate, Cyfluthrin, Cyhalothrin, Flucythrinate, Ethofenprox, Cycloprothrin, Tralomethrin, Silafluofen, Brofenprox, Acrinathrin, etc.
Bezoyl urea and other insecticides: Diflubenzuron, Chlorfluazuron, Hexaflumuron, Triflumuron, Tetrabenzuron, Fulfenoxuron, Flucycloxuron, Buprofezin, Pyriproxyfen, Methoprene, Benzoepin, Diafenthiuron, Imidacloprid, Fipronyl, Nicotin sulfate, Rotenone, Metaldehyde, Machine oil, Microbial insecticides such as BT and insect-pathogenic viruses, etc.
   Nematicides: Fenamiphos, Fosthiazate, etc.
   Acaricides:

Chlorbenzilate, Fenisobromolate, Dicofol, Amitraz, BPPS, Benzomate, Hexythiazox, Fenbutatin oxide, Polynactin, Quinomethionate, CPCBS, Tetradifon, Avermectin, Milbemectin, Clofentezin, Cyhexatin, Pyridaben, Fenpyroxymate, Tebufenpyrad, Pyrimidifen, Fenothiocarb, Dienochlor, etc.

Plant Groth Regulators: Gibberellins (e.g., Gibberellin A3, Gibberellin A4, Gibberellin A7), IAA, NAA, etc.

### EXAMPLES

The following examples serve to provide further appreciation of the invention but are not meant in any way to restrict the effective scope of the invention. The structures of isolated novel compounds were confirmed by NMR, Mass, and/or other appropriate analysis. examples for carrying out the formulations comprising the compound of the present invention will be explained. However, it should be noted that the type and incorporating rate of additives are not limited to those described in the following examples and may be modified over extensive range. Note that the term of “part” in the formulation example described below denotes “part by weight”.

### Example 1

### N-(5-Cloro-4-pentafluoroethylthiazol-2-yl)-3-trifluoromethylbenzamide

2-Amino-5-chloro-4-pentafluoroethylthiazole (0.4 g) was dissolved in pyridine (2 ml) and 3-trifluoromethylbenzoylchloride (0.35 g) was added at room temperature with stirring. The mixture was stirred for 3 hr at room temperature. The mixture was poured into ice water and acidified with aqueous hydrochloric acid then extracted with chloroform. The chloroform layer was dried over magnesium sulfate and the solvent was removed under reduced pressure. The solid thus obtained was recrystallized from n-hexane to give N-(5-chloro-4-pentafluoroethylthiazol-2-yl)-3-trifluoro-methylbenzamide (0.48 g), m. p. 156-157° C.

### Example 2

### N-(5-Iodo-4-pentafluoroethylthiazol-2-yl)-3,5-bis-trifluoro-methylbenzamide

2-Amino-5-iodo-4-pentafluoroethylthiazole (1 g) was dissolved in pyridine (3 ml) and 3,5-bis-trifluoromethylbenzoylchloride (0.8 g) was added at room temperature with stirring. The mixture was stirred for 2 hr at room temperature. The mixture was poured into ice water and acidified with aqueous hydrochloric acid then extracted with chloroform. The chloroform layer was dried over magnesium sulfate and the solvent was removed under reduced pressure. The solid thus obtained was recrystallized from methanol to give N-(5-iodo-4-pentafluoroethylthiazol-2-yl)-3,5-bis-trifluoromethyl-benzamide (0.55 g), m. p. 143-144° C.

### Example 3

### N-(5-Ethoxycarbonyl-4-pentafluoroethylthiazol-2-yl)-4-methylbenzamide

2-Amino-5-ethoxycarbonyl-4-pentafluoroethylthiazole (2 g) was dissolved in pyridine (3 ml) and p-toluoyl chloride (1.2 g) was added at room temperature with stirring. The mixture was stirred for 5 hr at room temperature. The mixture was poured into ice water and acidified with aqueous hydrochloric acid then extracted with chloroform. The chloroform layer was dried over magnesium sulfate and the solvent was removed under reduced pressure. The solid thus obtained was recrystallized from chloroform to give N-(5-ethoxycarbonyl-4-pentafluoroethylthiazol-2-yl)-4-methylbenzamide (2.4 g), m. p. 202-203° C.

### Example 4

### N-(5-Ethoxycarbonyl-4-pentafluoroethylthiazol-2-yl)-4-methylbenzamide

2-Amino-5-ethoxycarbonyl-4-pentafluoroethylthiazole (2 g) was dissolved in pyridine (3 ml) and p-toluoyl chloride (1.2 g) was added at room temperature with stirring. The mixture was stirred for 5 hr at room temperature. The mixture was poured into ice water and acidified with aqueous hydrochloric acid then extracted with chloroform. The chloroform layer was dried over magnesium sulfate and the solvent was removed under reduced pressure. The solid thus obtained was recrystallized from carbon tetrachloride to give N-(5-ethoxycarbonyl-4-pentafluoroethylthiazol-2-yl)-4-methylbenzamide (2.4 g), m. p. 202-203° C.

### Example 5

### N-(5-Ethoxycarbonyl-4-pentafluoroethylthiazol-2-yl)-3,5-bis-trifluoromethylbenzamide

2-Amino-5-ethoxycarbonyl-4-pentafluoroethylthiazole (4 g) was dissolved in pyridine (10 ml) and 3,5-bis-trifluoromethylbenzoyl chloride (3.9 g) was added at room temperature with stirring. The mixture was stirred for 5 hr at room temperature. The mixture was poured into ice water and acidified with aqueous hydrochloric acid then extracted with chloroform. The chloroform layer was dried over magnesium sulfate and the solvent was removed under reduced pressure. The solid thus obtained was recrystallized from n-hexane-carbon tetrachloride to give N-(5-ethoxycarbonyl-4-pentafluoroethylthiazol-2-yl)-3,5-bis-trifluoromethyl-benzamide (7.3 g), m. p. 154-155° C.

### Example 6

### N-(5-carboxy-4-pentafluoroethylthiazol-2-yl)-3,5-bis-trifluoromethylbenzamide

N-(5-Ethoxycarbonyl-4-pentafluoroethylthiazol-2-yl)-3,5-bis-trifluoromethyl-benzamide (7 g) was dissolved in tetrahydrofuran (20 ml) and sodium hydroxide (1.5 g) in water (10 ml) and ethanol (10 ml) were added at room temperature. The reaction mixture was stirred for 1 day at room temperature. The solvent was removed under reduced pressure and then acidified with diluted hydrochloric acid. The precipitated N-(5-carboxy-4-pentafluoroethylthiazol-2-yl)-3,5-bis-trifluoromethylbenzamide (6.7 g, m. p. 248-249° C.) was filtered and well dried.

### Example 7

### N-(5-Chlorocarbonyl-4-pentafluoroethylthiazol-2-yl)-3,5-bis-tri-fluoromethybenzamide

N-(5-carboxy-4-pentafluoroethylthiazol-2-yl)-3,5-bis-trifluoromethylbenzamide (6.7 g) was mixed with thionyl chloride (15 ml) and benzene (15 ml) and then refluxed for 3 hr with stirring. The solvent and excess thionyl chloride were removed under reduced pressure to give white crystalline N-(5-chlorocarbonyl-4-pentafluoroethylthiazol-2-yl)-3,5-bis-trifluoromethybenz-amide (7 g, m. p. 164-165° C.).

### Example 8

### N-(5-Hydroxymethyl-4-pentafluoroethylthiazol-2-yl)-3,5-bis-trifluoromethylbenzamide

N-(5-Chlorocarbonyl-4-pentafluoroethylthiazol-2-yl)-3,5-bis-trifluoromethyl-benzamide (7 g) was dissolved in dioxane (50 ml) and sodium borohydride (2 g) was added at room temperature. The mixture was refluxed for 3 hr with stirring. It was cooled down to 10° C. and cold water (30 ml) was added little by little then 3-N hydrochloric acid (30 ml). After adding additional water (100 ml), the precipitated solids were extracted with chloroform. Purification of the crude substance by silicagel column chromatography (chloroform-2.5% methanol as an eluent) followed by recrystallization from carbon tetrachloride afforded N-(5-hydroxymethyl-4-pentafluoroethylthiazol-2-yl)-3,5-bis-trifluoromethylbenzamide (3.89 g, m. p. 164-165° C.).

### Example 9

### N-(5-chloromethyl-4-pentafluoroethylthiazol-2-yl)-3,5-bis-trifluoromethylbenzamide

N-(5-Hydroxymethyl-4-pentafluoroethylthiazol-2-yl)-3,5-bis-trifluoromethylbenzamide (0.8 g) was mixed with thionyl chloride (1 ml) and benzene (5 ml). The mixture was refluxed for 3 hr then poured into ice water and extracted with benzene. The extract was dried over magnesium sulfate and the solvent was removed under reduced pressure. After recrystallization from carbon tetrachloride-n-hexane, N-(5-fluoromethyl-4-pentafluoroethylthiazol-2-yl)-3,5-bis-trifluoromethylbenzamide (0.7 g, m. p. 133-134° C.) was obtained.

### Example 10

### N-(4-n-heptafluoropropyl-5-iodothiazol-2-yl)-3-trifluoromethylbenzamide

2-Amino-4-n-heptafluoropropylthiazole (2 g) was dissolved in pyridine (3 ml) and 3-trifluoromethylbenzoylchloride (1.1 g) was added at room temperature with stirring. The mixture was stirred for 3 hr at room temperature. The mixture was poured into ice water and acidified with aqueous hydrochloric acid then extracted with chloroform. The chloroform layer was dried over magnesium sulfate and the solvent was removed under reduced pressure. The residue was purified by silicagel column chromatography (chloroform-n-hexane 1:1 as an eluent) and the solid thus obtained was recrystallized from n-hexane to give N-(4-n-heptafluoropropylthiazol-2-yl)-3-trifluoro-methylbenzamide (0.87 g), m. p. 96-98° C.

### Example 11

### N-(4-n-Heptafluoropropyl-5-iodothiazol-2-yl)-3-trifluoromethylbenzimidoyl chloride

N-(4-n-Heptafluoropropyl-5-iodothiazol-2-yl)-3-trifluoromethylbenzamide (0.95 g) was mixed with phosphorous oxychloride (5 ml) and phosphorous pentachloride (0.35 g) and the mixture was refluxed for 4 hr. The excess phosphorous oxychloride was removed under reduced pressure. The residue was added toluene (50 ml) and the solvent was removed again under reduced pressure. This was used to the next reaction without further purification.

### Example 12

### N-(4-n-Heptafluoropropyl-5-iodothiazol-2-yl)-3-trifluoromethylbenzthioamide

N-(4-n-Heptafluoropropyl-5-iodothiazol-2-yl)-3-trifluoromethylbenzimidoyl chloride (crude, 1 g) was mixed with thiourea (1 g) in tetrahydrofuran (10 ml) and refluxed for 3 hrs. The solvent was removed under reduced pressure and the residue was extracted with chloroform. The extract was dried over magnesium sulfate and the solvent was removed under reduced pressure. The residue was purified by silicagel column chromatography (chloroform-n-hexane 1:1 as an eluent) and recrystallized from n-hexane to give N-(4-n-heptafluoropropyl-5-iodothiazol-2-yl)-3-trifluoromethylbenzthioamide (0.48 g, m. p. 97-98° C.) as slightly yellow crystals.

### Example 13

### Wettable powder formulation

| | |
|---|---|
| A compound of present invention | 40 part |
| Diatomaceous earth | 53 part |
| Higher alcohol sulfate | 4 part |
| Alkylnaphthalenesulfonate | 3 part |

The components given above are mixed and pulverized to fine particles to thereby give a wettable powder formulation for the compound of the present invention with the content of 40% based on the active ingredient.

### Example 14

### Emulsifiable Concentrate Formulation

| | |
|---|---|
| A compound of present invention | 30 part |
| Xylene | 33 part |
| Dimethylformamide | 30 part |
| Polyoxyethylene alkyl allyl ether | 7 part |

The components given above are mixed and prepared to a solution to thereby give an emulsifiable concentrate formulation for the compound of the present invention with the content of 30% based on the active ingredient.

### Example 15

### Dust Formulation

| | |
|---|---|
| A compound of present invention | 10 part |
| Talc | 89 part |
| Polyoxyethylene alkyl allyl ether | 1 part |

The components given above are mixed and pulverized to fine particles to thereby give a dust formulation for the compound of the present invention with the content of 10% based on the active ingredient.

### Example 16

### Granular Formulation

| | |
|---|---|
| A compound of present invention | 5 part |
| Clay | 73 part |
| Bentonite | 20 part |
| Dioctylsulfosuccinate sodium salt | 1 part |
| Sodium phosphate | 1 part |

The components given above are mixed, thoroughly ground, added with water, then kneaded, and granulated, and further dried to thereby give a granular formulation for the compound of the present invention with the content of 5% based on the active ingredient.

### Example 17

### Suspension Concentrate Formulation

| | |
|---|---|
| A compound of present invention | 10 part |
| Sodium ligninsulfonate | 4 part |
| Sodium dodecylbenzenesulfonate | 1 part |
| Xanthane gum | 0.2 part |
| Water | 84.8 part |

The components given above are mixed and ground by wet grinding to a particle size of less than 1 μm to thereby give a suspension concentrate for the compound of the present invention with the content of 10% based on the active ingredient.

### Example 18

### Efficacy against Tetranychus urticae (Tu; Two-Spotted Spider Mites:

Fifteen adult females were inoculated on a potted Kidney-bean seedling. On the next day, the chemical solution at 125 ppm was sprayed on the Kidney-bean seedling by a glass nozzle using an air compressor. After air-dried the Kidney-bean pot was maintained in a room kept at 25° C. and 65% RH.

On three days after treatment, the numbers of dead and alive mites were counted and mortality was calculated by Abott's formula.

Each test was duplicated.
Abbott's formula:

$$mortality = \frac{\begin{matrix}Survial rate in untreated polt - \\ Survival rate in treated plot\end{matrix}}{Survial rate in untreated polt} \times 100$$

The following compounds showed 100% control.
3, 18, 44, 51, 72, 77, 114, 124, 129, 148, 185, 211.

### Example 19

### Efficacy against Pseudaletia separata Walker (Ps; rice armyworm)

A piece of Maize leaf (ca.7×0.5 cm) was dipped in the chemical solution at 125 ppm for 30 sec and air-dried. The leaf was placed in a glass petri dish (9 cm diameter) and five second-instar larvae of Pseudaletia separata were introduced into the petri dish. Five days after the treatment, the number of survival larvae was counted and the mortality was calculated by Abott's formula. Each test was duplicated.

The following compounds showed 100% control.

3, 16, 18, 28, 30, 38, 39, 41, 46, 51, 72, 77, 79, 103, 108, 114, 122, 124, 129, 131, 133, 148, 149, 152, 155, 156, 178, 188, 193, 194, 209, 211, 222.

TABLE 1

| Compound No. | R1 | R2 | R3 | R4 | X | m. p. |
|---|---|---|---|---|---|---|
| 1 | C2F5 | Cl | H | 2-Cl-4-SO2Me—C6H3 | O | |
| 2 | C2F5 | CO2Et | H | 4-Me—C6H4 | O | 202-203 |
| 3 | C2F5 | Br | H | 4-CF3—C6H4 | O | 145-147 |
| 4 | C2F5 | Cl | H | 4-SO2CF3—C6H4 | O | |
| 5 | i-C3F7 | I | Me | 3,4-Cl2—C6H3 | S | |
| 6 | i-C3F7 | I | CH2OEt | 3-Cl-4-CF3—C6H3 | S | |
| 7 | C2F5 | Br | H | 4-EtOCO2—C6H4 | O | |
| 8 | C2F5 | Br | H | 4-NMe2OCO—C6H4 | O | |
| 9 | n-C3F7 | CH═CHNO2 | H | 3-CF3—C6H4 | S | |
| 10 | C2F5 | Br | H | 4-OAc—C6H4 | O | |
| 11 | C2F5 | Br | H | 3,4-methylenedioxy-C6H4 | O | |
| 12 | C2F5 | Br | H | 4-Ac—C6H4 | O | |
| 13 | C2F5 | Cl | H | 2,3-Cl2-4-CF3—C6H2 | O | |
| 14 | C2F5 | Cl | H | 4-NO2—C6H4 | O | |
| 15 | C2F5 | Cl | H | 2-Br-4-CO2Et—C6H3 | O | |
| 16 | C2F5 | I | H | 3-CF3—C6H4 | O | 141-143 |
| 17 | C2F5 | I | H | 2,6-F2—C6H3 | O | |
| 18 | C2F5 | Cl | H | 3-CF3—C6H4 | O | 156-157 |
| 19 | i-C3F7 | 3-CF3—C6H4 | H | 3-CF3—C6H4 | O | |
| 20 | C2F5 | CH2SCN | H | 3,5-(CF3)2—C6H3 | O | oil |
| 21 | C2F5 | CO2Et | H | 3,5-(CF3)2—C6H3 | O | 144-145 |
| 22 | n-C3F7 | CH2OMe | H | 3-CF3—C6H4 | O | |
| 23 | C2F5 | 4-C6H5—C6H4 | H | 3,5-Me2—C6H3 | O | oil |
| 24 | i-C3F7 | Cl | H | 2-Cl-4,5-F2—C6H2 | O | 92-93 |
| 25 | C2F5 | CO2Et | H | 3-CF3—C6H4 | O | 93-94 |
| 26 | C2F5 | Cl | H | 2,4-F2—C6H3 | O | |
| 27 | n-C3F7 | Cl | H | 4-i-C3F7—C6H4 | O | 136-137 |
| 28 | i-C3F7 | H | H | 3-CF3—C6H4 | O | oil |
| 29 | C2F5 | I | H | 4-SO2Me—C6H4 | O | |
| 30 | n-C3F7 | H | H | 3-i-C3F7—C6H4 | O | 125-126 |
| 31 | i-C3F7 | F | H | 4-F—C6H4 | O | |
| 32 | C2F5 | I | H | 2,4-Me2—C6H3 | O | |
| 33 | C2F5 | CH2OH | H | 3,5-Me2—C6H3 | O | 157-158 |
| 34 | i-C3F7 | I | H | 1-naphthyl | O | |
| 35 | i-C3F7 | I | H | 2,6-F2—C6H3 | O | |
| 36 | C2F5 | H | H | 3-SO2CF3—C6H4 | O | |
| 37 | i-C3F7 | Cl | H | 3,4-Cl2—C6H3 | S | |
| 38 | i-C3F7 | Br | H | 2,4-F2—C6H3 | S | oil |
| 39 | C2F5 | Br | H | 3-CF3—C6H4 | O | 134-135 |
| 40 | C2F5 | 4-Br-styryl | H | 3,5-Cl2—C6H3 | O | |
| 41 | C2F5 | H | CH2OEt | 3-CF3—C6H4 | O | oil |
| 42 | C2F5 | Br | H | 2,6-Cl2—C6H3 | O | |
| 43 | C2F5 | CH2Cl | H | 3-Cl—C6H4 | O | 65-68 |
| 44 | C2F5 | I | H | 3,5-(CF3)2—C6H3 | O | 143-144 |
| 45 | i-C3F7 | CH2SMe | H | 3-Cl—C6H4 | O | |
| 46 | C2F5 | Br | H | 4-CF3-3-pyridinyl | O | 166-168 |
| 47 | C2F5 | CH2Cl | H | 3,5-(CF3)2—C6H3 | O | 133-134 |
| 48 | C2F5 | H | H | 4-OMe—C6H4 | O | |
| 49 | n-C3F7 | Br | H | 3-CF3—C6H4 | S | |
| 50 | C2F5 | Br | H | 4-Br—C6H4 | O | |
| 51 | C2F5 | Br | H | 3,5-(CF3)2—C6H3 | O | 139-140 |
| 52 | C2F5 | CHO | H | 2,4-Cl2C6H3 | O | |
| 53 | C2F5 | H | H | 3,5-(CF3)2—C6H3 | O | oil |
| 54 | C2F5 | 4-Me-styryl | H | 4-SO2Me—C6H4 | O | |
| 55 | C2F5 | Br | H | 3-OCF3—C6H4 | O | |
| 56 | C2F5 | F | H | 2,6-OMe2—C6H3 | O | |
| 57 | i-C3F7 | Br | H | 3,5-(CF3)2—C6H3 | O | |
| 58 | C2F5 | Br | H | 2,4-F2—C6H3 | O | |
| 59 | C2F5 | CH2F | H | 3,5-(CF3)2—C6H3 | O | amorphous |
| | | | | | | solid |
| 60 | C2F5 | I | H | 1-C6H5-5-CF3-pyrazol-4-yl | O | |
| 61 | i-C3F7 | 3-CF3—C6H4 | H | 3-CF3—C6H4 | S | |
| 62 | C2F5 | Cl | H | 4-OCO2EtC6H4 | O | |
| 63 | i-C3F7 | I | H | 4-CF3—C6H4 | O | 182-183 |
| 64 | i-C3F7 | Br | H | 3-pyridinyl | O | |
| 65 | C2F5 | CH2SCN | Me | 4-CF3—C6H4 | O | oil |
| 66 | C2F5 | I | H | 2,4,6-Cl3—C6H2 | O | |
| 67 | C2F5 | H | H | 4-NMe2—C6H4 | O | |
| 68 | C2F5 | CO2Et | H | 3,5-(CF3)2—C6H3 | O | 154-155 |
| 69 | C2F5 | Cl | OEt | 3-CF3—C6H4 | O | |
| 70 | | | | | | |
| 71 | | | | | | |
| 72 | C2F5 | CN | H | 3-CF3—C6H4 | O | 165-166 |
| 73 | i-C3F7 | 4-Cl—C6H4 | H | 3-CF3—C6H4 | O | |
| 74 | C2F5 | Cl | H | 3-SOEt—C6H4 | O | |
| 75 | i-C3F7 | I | H | 3-pyridinyl | O | |
| 76 | C2F5 | I | H | 2,4-Cl2—C6H3 | O | |
| 77 | i-C3F7 | Cl | H | 3-CF3—C6H4 | O | 146-147 |
| 78 | C2F5 | Br | H | 4-Cl-3-F—C6H3 | O | |
| 79 | i-C3F7 | Cl | H | 3-i-C3F7—C6H4 | O | 191-192 |
| 80 | i-C3F7 | Cl | H | 4-C6H4—C6H4 | O | |
| 81 | C2F5 | Cl | H | 2,4-Cl2—C6H3 | O | |
| 82 | C2F5 | Cl | H | 2,4,6-Me3—C6H2 | O | |
| 83 | C2F5 | 4-CF3-styryl | H | 3-CF3—C6H4 | O | 96-97 |
| 84 | C2F5 | I | H | 2,6-Cl2—C6H3 | O | |
| 85 | n-C3F7 | CH2SEt | H | 4-Cl—C6H4 | O | |
| 86 | C2F5 | CH2OH | H | 4-CF3—C6H4 | O | |
| 87 | C2F5 | CH2Cl | H | 4-Me—C6H4 | O | 185-186 |
| 88 | i-C3F7 | I | H | 3,5-(CF3)2—C6H3 | O | |
| 89 | C2F5 | Br | H | 4-Cl-2-OMe—C6H3 | O | |
| 90 | C2F5 | CH2OMe | H | 3,5-(CF3)2—C6H4 | O | oil |
| 91 | C2F5 | Cl | O-allyl | 3-CF3—C6H4 | O | |
| 92 | C2F5 | Br | H | 2,6-(OMe)2—C6H3 | O | |
| 93 | C2F5 | CH2I | Me | 3-CF3—C6H4 | S | 67-70 |
| 94 | C2F5 | Cl | H | 2,6-Cl2—C6H3 | O | |
| 95 | n-C3F7 | Cl | CH2OEt | 2-Cl-4,5-F2—C6H3 | O | oil |
| 96 | C2F5 | Cl | O-propargyl | 3-CF3—C6H4 | O | |
| 97 | C2F5 | I | H | 2-Cl-4-SO2Me—C6H3 | O | |
| 98 | n-C3F7 | Cl | H | 3,4-Cl2—C6H3 | O | |
| 99 | n-C3F7 | CH═NOMe | H | 3,5-(CF3)2—C6H3 | O | 171-174 |
| 100 | C2F5 | H | H | 2-furyl | O | |
| 101 | n-C3F7 | Br | H | 4-AcOCH2—C6H4 | O | |
| 102 | C2F5 | I | H | C6H5 | O | 122-123 |
| 103 | C2F5 | 4-F—C6H4 | H | 4-CF3—C6H4 | O | 253-254 |
| 104 | C2F5 | 3,4-Cl2-styryl | H | 3-CF3—C6H4 | O | |
| 105 | C2F5 | CH2S—Pr-i | H | 3-Cl—C6H4 | O | oil |
| 106 | C2F5 | H | OMe | 3-CF3—C6H4 | O | |
| 107 | C2F5 | CH2F | H | 3,5-Me2—C6H3 | O | 127-128 |
| 108 | C2F5 | H | H | 3-CF3—C6H4 | S | 63-66 |
| 109 | C2F5 | I | H | 4-CO2Et—C6H4 | O | |
| 110 | C2F5 | CH2Cl | H | 3-CF3—C6H4 | O | 106-107 |
| 111 | C2F5 | Br | H | 3,5-Me2—C6H3 | O | 152-153 |
| 112 | C2F5 | I | H | 2-Me-3-CF3—C6H3 | O | |
| 113 | n-C3F7 | 4-CN-styryl | H | 4-F—C6H4 | O | |
| 114 | C2F5 | Cl | H | 3,5-(CF3)2—C6H3 | O | 164-165 |
| 115 | C2F5 | I | H | 4-t-Bu—C6H4 | O | |
| 116 | C2F5 | F | H | 4-Cl—C6H4 | O | |
| 117 | C2F5 | CH2SCN | H | 4-Me—C6H4 | O | 176-177 |
| 118 | n-C3F7 | 4-NO2-styryl | H | 4-SO2CF3—C6H4 | O | |
| 119 | C2F5 | I | H | 3,4-Cl2—C6H3 | S | |
| 120 | C2F5 | CH2OH | H | 3-CF3—C6H4 | O | 192-193 |
| 121 | C2F5 | Cl | Me | 2,4-Cl2—C6H3 | O | |
| 122 | C2F5 | H | H | 3-OCF3—C6H4 | O | oil |
| 123 | C2F5 | 2,6-Cl2-styryl | H | 3-CF3—C6H4 | O | |
| 124 | C2F5 | Cl | H | 4-CF3—C6H4 | O | 134-136 |
| 125 | i-C3F7 | Br | H | 3-CF3—C6H4 | O | 157-158 |
| 126 | C2F5 | CH2OAc | H | 3,5-(CF3)2—C6H4 | O | 127-128 |
| 127 | C2F5 | I | H | 4-CN—C6H4 | O | |
| 128 | C2F5 | 4-Me-styryl | i-Bu | 3-Cl—C6H4 | O | 102-105 |
| 129 | C2F5 | Cl | H | 3-i-C3F7—C6H4 | O | 142-143 |
| 130 | n-C3F7 | CH═CHNO2 | H | 4-CF3—C6H4 | O | |
| 131 | C2F5 | I | H | 4-i-C3F7—C6H4 | O | 132-133 |
| 132 | C2F5 | Cl | CH2OEt | 3-CF3—C6H4 | O | oil |
| 133 | C2F5 | H | H | 4-i-C3F7—C6H4 | O | oil |
| 134 | C2F5 | Cl | H | 3-Br—C6H4 | O | 147-149 |
| 135 | C2FS | Br | H | 3-thienyl | O | |
| 136 | C2F5 | 2,6-F2-styryl | H | 3-CF3—C6H4 | O | 127-128 |
| 137 | C2F5 | I | H | 2-CF3—C6H4 | O | |
| 138 | C2F5 | Br | H | 4-C6H5—C6H4 | O | 168-170 |
| 139 | n-C3F7 | CH═C(CN)2 | H | 3,5-(CF3)2—C6H3 | O | 175-176 |
| 140 | n-C3F7 | Cl | H | 3-Cl—C6H4 | O | |
| 141 | C2F5 | H | H | 3-i-Pr—C6H4 | O | |
| 142 | C2F5 | CHO | H | 3-CF3—C6H4 | O | |
| 143 | n-C3F7 | H | H | 4-i-C3F7—C6H4 | O | 67-69 |
| 144 | n-C3F7 | I | H | 4-i-C3F7—C6H4 | O | 126-128 |
| 145 | C2F5 | Cl | H | 4-i-C3F7—C6H4 | O | 149-150 |
| 146 | n-C3F7 | Cl | H | 3,5-(CF3)2—C6H3 | O | |
| 147 | C2F5 | CH═CH—CN | H | 3-Cl—C6H4 | O | |
| 148 | C2F5 | Cl | H | 2-Cl-4,5-F2—C6H2 | O | oil |
| 149 | C2F5 | I | H | 3,4-Cl2—C6H3 | O | 159-161 |
| 150 | C2F5 | Cl | H | 4-COMe—C6H4 | O | |
| 151 | C2F5 | Br | H | 4-NH2—C6H4 | O | |
| 152 | C2F5 | H | H | 3,4-Cl2—C6H3 | O | 164-167 |
| 153 | C2F5 | I | H | 3-OCF3—C6H4 | O | 114-115 |
| 154 | C2F5 | I | H | 3-Cl—C6H4 | N—O-allyl | |
| 155 | n-C3F7 | I | H | 3-CF3—C6H4 | O | 96-98 |
| 156 | n-C3F7 | H | H | 3-CF3—C6H4 | O | 106-108 |
| 157 | n-C3F7 | I | H | 3,5-(CF3)2—C6H3 | O | |
| 158 | C2F5 | CH═N—O—CH2-3,4-Cl2—C6H3 | H | 3-CF3—C6H4 | O | |
| 159 | C2F5 | 3-OMe-styryl | H | 3-F—C6H4 | O | |
| 160 | n-C3F7 | I | Ac | 3-CF3—C6H4 | O | |
| 161 | C2F5 | Br | H | 4-EtOCOCH2—C6H4 | O | |
| 162 | C2F5 | Br | H | 4-EtOCOCH2O—C6H4 | O | |
| 163 | C2F5 | Br | H | 4-CH2Cl—C6H4 | O | |
| 164 | C2F5 | Br | H | 4-CH2CH═CH2—C6H4 | O | |
| 165 | C2F5 | Br | H | 4-ethenyl-C6H4 | O | |
| 166 | n-C3F7 | I | H | 3,5-Me2—C6H3 | O | 156-157 |
| 167 | C2F5 | 4-Br-styryl | H | 3-CF3—C6H4 | O | |
| 168 | n-C3F7 | I | Et | 3-CF3—C6H4 | O | |
| 169 | C2F5 | I | OEt | 3-CF3—C6H4 | O | |
| 170 | n-C3F7 | Cl | Me | 2,4-Cl2—C6H3 | S | |
| 171 | n-C3F7 | I | H | 3-Cl—C6H4 | S | 97-98 |
| 172 | C2F5 | I | H | 4-Br-2-F—C6H3 | O | |
| 173 | n-C3F7 | Cl | H | 2,4-F2—C6H3 | O | |
| 174 | C2F5 | I | H | 2,4-F2—C6H3 | NOEt | |
| 175 | n-C3F7 | Cl | H | 4-t-Bu—C6H4 | O | |
| 176 | n-C3F7 | I | H | 2,4,6-Me3—C6H2 | O | |
| 177 | C2F5 | H | H | 4-SMe—C6H4 | O | |
| 178 | n-C3F7 | Cl | H | 3-i-C3F7—C6H4 | O | 150-151 |
| 179 | C2F5 | Br | OEt | 3-CF3—C6H4 | O | |
| 180 | n-C3F7 | Cl | H | 3,5-Cl2—C6H3 | O | |
| 181 | C2F5 | SO2Me | H | 3-CF3—C6H4 | O | 208-210 |
| 182 | i-C3F7 | CN | H | 2,6-Cl2-4-SO2Me—C6H2 | O | |
| 183 | n-C3F7 | 3,5-Me2—C6H3 | H | 3-CF3—C6H4 | O | 157-158 |
| 184 | n-C3F7 | I | Et | 3-CF3—C6H4 | O | |
| 185 | n-C3F7 | Cl | H | 4-Cl-2,5-F2—C6H2 | O | 118-119 |
| 186 | C2F5 | I | H | 3-CF3—C6H4 | N—OMe | 92-93 |
| 187 | i-C3F7 | Cl | H | 4-Cl—C6H4 | S | |
| 188 | C2F5 | H | H | 3-CF3—C6H4 | N—OMe | oil |
| 189 | C2F5 | Br | H | 3-Cl—C6H4 | O | 132-133 |
| 190 | n-C3F7 | I | CH2OMe3—CF3—C6H4 | | S | |
| 191 | i-C3F7 | I | H | 3-CF3—C6H4 | O | 197-199 |
| 192 | n-C3F7 | Br | Et | 2-Cl-4-NO2—C6H3 | S | |
| 193 | C2F5 | I | H | 3-CF3—C6H4 | S | 73-75 |
| 194 | C2F5 | I | CH2OEt | 3-CF3—C6H4 | O | oil |
| 195 | i-C3F7 | Cl | H | 3-OCF3—C6H4 | O | |
| 196 | C2F5 | CH2OH | H | 3-Cl—C6H4 | O | 202-203 |
| 197 | C2F5 | Br | H | 4-CN-3-CF3—C6H3 | O | |
| 198 | C2F5 | Br | H | 2-OMe-4-CF3—C6H3 | O | |
| 199 | n-C3F7 | CH2OH | H | 4-F—C6H4 | S | |
| 200 | C2F5 | Br | H | 2-Cl-4-CF3—C6H3 | O | |
| 201 | C2F5 | Br | H | 4-C2F5—C6H4 | O | |
| 202 | C2F5 | Br | H | 3-Cl-5-CF3—C6H3 | O | |
| 203 | C2F5 | Br | H | 2-F-4-Br—C6H3 | O | |
| 204 | C2F5 | CH2OH | H | 3,5-(CF3)2—C6H3 | O | 127-128 |
| 205 | C2F5 | 4-C6H5O—C6H4 | H | 4-Me—C6H4 | O | |
| 206 | C2F5 | 4-CF3—C6H4 | H | 3,4-Cl2—C6H3 | O | |
| 207 | C2F5 | Br | H | 4-C6H4O—C6H4 | O | |
| 208 | i-C3F7 | Cl | H | C6H5 | O | |
| 209 | C2F5 | H | H | 3-CF3—C6H4 | O | 86-87 |
| 210 | C2F5 | CHO | H | 3-CF3—C6H4 | O | 177-179 |
| 211 | n-C3F7 | Cl | H | 3-CF3—C6H4 | O | 156-157 |
| 212 | C2F5 | I | H | 4-NO2—C6H4 | O | |
| 213 | i-C3F7 | Cl | H | 1-naphthyl | S | |
| 214 | i-C3F7 | I | H | 3-i-C3F7—C6H4 | O | 200-201 |
| 215 | C2F5 | I | H | 4-C6H4—C6H4 | S | |
| 216 | C2F5 | CH2SCN | H | 3-Cl—C6H4 | O | 163-164 |
| 217 | C2F5 | CH═NOEt | H | 3-CF3—C6H4 | O | oil |
| 218 | C2F5 | CH═CHNO2 | H | 3-CF3—C6H4 | O | 139-141 |
| 219 | n-C3F7 | 4-SO2Me—C6H4 | H | 4-CF3—C6H4 | S | |
| 220 | C2F5 | CO2Et | H | 3-Cl—C6H4 | O | 143-144 |
| 221 | C2F5 | Br | H | 4-Cl-2-Me—C6H3 | O | |
| 222 | C2F5 | H | H | 4-CF3—C6H4 | O | 128-129 |
| 223 | i-C3F7 | I | H | 5-I-4-CF3-thiazol-2-yl | O | |
| 224 | C2F5 | CH═CH—CN | H | 3-CF3—C6H4 | O | 170-172 |
| 225 | C2F5 | SMe | H | 3-CF3—C6H4 | O | 158-160 |
| 226 | C2F5 | Cl | H | 4-NMe2—C6H4 | O | |
| 227 | C2F5 | Cl | H | 2,6-OMe2—C6H3 | O | |
| 228 | C2F5 | I | H | 4-Cl—C6H4 | O | |
| 229 | i-C3F7 | Cl | H | 2-Cl-4-OMe—C6H3 | O | |
| 230 | n-C3F7 | Br | H | 4-CF3—C6H4 | S | |
| 231 | C2F5 | I | H | 3,5-Cl2—C6H3 | O | |
| 232 | C2F5 | I | H | 2,3,5-F3—C6H2 | O | |
| 233 | C2F5 | I | H | 2-Cl-4-OMe—C6H3 | O | |
| 234 | C2F5 | I | H | 2-NO2-4-SO2Me—C6H3 | O | |
| 235 | C2F5 | Cl | H | 2,3,5,6-F4—C6H | O | |
| 236 | C2F5 | Cl | H | 4-CN—C6H4 | O | |
| 237 | C2F5 | Cl | H | 3-SO2Me—C6H4 | O | |
| 238 | C2F5 | Cl | H | 3,5-Cl2—C6H3 | O | |
| 239 | C2F5 | CHF2 | H | 3-CF3—C6H4 | O | 112-113 |
| 240 | C2F5 | Br | H | 2,4-Me2—C6H3 | O | |
| 241 | C2F5 | Br | H | 2-SO2Me,4-CF3—C6H3 | O | |
| 242 | C2F5 | Br | H | 3,4-(CF3)2—C6H3 | O | |
| 243 | C2F5 | CH═C(CN)2 | H | 3-CF3—C6H4 | O | 253-254 |
| 244 | i-C3F7 | 4-F—C6H4 | H | 4-F—C6H4 | O | |
| 245 | n-C3F7 | F | H | 4-Br—C6H4 | S | |
| 246 | i-C3F7 | CHO | H | 3,4-Cl2—C6H3 | S | |
| 247 | C2F5 | CH═NOMe | H | 3-CF3—C6H4 | O | 117-118 |

1. The use as an insecticide and/or acaricide of a compound of formula (1): 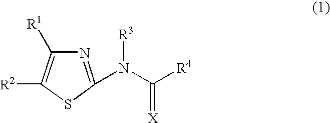
wherein
R¹ is C₂F₅, n-C₃F₇, i-C₃F₇,
R² is H, halogen, cyano, alkoxycarbonyl, hydroxymethyl, haloalkyl, alkylthioalkyl, alkoxyalkyl, acyloxyalkyl, formyl, thiocyanatoalkyl, alkylsulfonyl, alkylthio, alkoxyiminomethyl, benzyloxyiminomethyl being optionally substituted by one or more of halogen, alkyl, alkoxy, cyano, nitro, vinyl being optionally substituted by one or more halogen, nitro or cyano, styryl being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy, or phenyl being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy; thiocyanatoalkyl,
R³ is H, alkyl, alkoxyalkyl or alkoxy,
R⁴ is aryl (especially phenyl, naphthyl, pyridinyl, pyrimidinyl, thienyl, furyl, thiazolyl, isothiazolyl, pyrazolyl, oxazolyl, isoxazolyl, thiadiazolyl (1,2,4, and 1,3,4), oxadiazolyl (1,2,4- and 1,3,4); being optionally substituted by one or more of halogen, cyano, alkyl, haloalkyl, alkoxy, nitro, alkoxycarbonyl, alkylcarbonyloxy, alkylcarbonyl, amino, alkylamino, haloalkoxy, alkylthio, or alkylsulfonyl, haloalkylsulfonyl), X is O, S or NR⁵, and
R⁵ is alkyl, cycloalkyl, alkoxy, alkenylalkyloxy or alkynylalkyloxy.

2. The use of a composition comprising an insecticidally or acaricidally effective amount of a compound of formula (1) 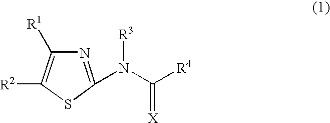
wherein
R¹ is C₂F₅, n-C₃F₇, i-C₃F₇,
R² is H, halogen, cyano, alkoxycarbonyl, hydroxymethyl, haloalkyl, alkylthioalkyl, alkoxyalkyl, acyloxyalkyl, formyl, thiocyanatoalkyl, alkylsulfonyl, alkylthio, alkoxyiminomethyl, benzyloxyiminomethyl being optionally substituted by one or more of halogen, alkyl, alkoxy, cyano, nitro, vinyl being optionally substituted by one or more halogen, nitro or cyano, styryl being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy, or phenyl being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy; thiocyanatoalkyl,
R³ is H, alkyl, alkoxyalkyl or alkoxy,
R⁴ is aryl (especially phenyl, naphthyl, pyridinyl, pyrimidinyl, thienyl, furyl, thiazolyl, isothiazolyl, pyrazolyl, oxazolyl, isoxazolyl, thiadiazolyl (1,2,4, and 1,3,4), oxadiazolyl (1,2,4- and 1,3,4); being optionally substituted by one or more of halogen, cyano, alkyl, haloalkyl, alkoxy, nitro, alkoxycarbonyl, alkylcarbonyloxy, alkylcarbonyl, amino, alkylamino, haloalkoxy, alkylthio, or alkylsulfonyl, haloalkylsulfonyl), X is O, S or NR⁵, and
R⁵ is alkyl, cycloalkyl, alkoxy, alkenylalkyloxy or alkynylalkyloxy; and
a carrier or diluent to combat and control insect pests at a locus.

3. A compound of formula (1): 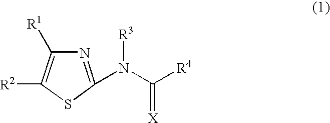
wherein
R¹ is C₂F₅, n-C₃F₇, i-C₃F₇,
R² is H, halogen, cyano, alkoxycarbonyl, hydroxymethyl, haloalkyl, alkylthioalkyl, alkoxyalkyl, acyloxyalkyl, formyl, thiocyanatoalkyl, alkylsulfonyl, alkylthio, alkoxyiminomethyl, benzyloxyiminomethyl being optionally substituted by one or more of halogen, alkyl, alkoxy, cyano, nitro, vinyl being optionally substituted by one or more halogen, nitro or cyano, styryl being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy, or phenyl being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy; thiocyanatoalkyl,
R³ is H, alkyl, alkoxyalkyl or alkoxy,
R⁴ is aryl (especially phenyl, naphthyl, pyridinyl, pyrimidinyl, thienyl, furyl, thiazolyl, isothiazolyl, pyrazolyl, oxazolyl, isoxazolyl, thiadiazolyl (1,2,4, and 1,3,4), oxadiazolyl (1,2,4- and 1,3,4); being optionally substituted by one or more of halogen, cyano, alkyl, haloalkyl, alkoxy, nitro, alkoxycarbonyl, alkylcarbonyloxy, alkylcarbonyl, amino, alkylamino, haloalkoxy, alkylthio, or alkylsulfonyl, haloalkylsulfonyl), X is O, S or NR¹, and
R⁵ is alkyl, cycloalkyl, alkoxy, alkenylalkyloxy or alkynylalkyloxy.

4. An insecticidal or acaricidal composition comprising a compound of formula (1): 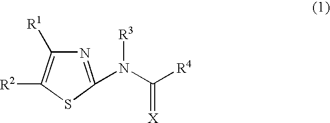
wherein
R¹ is C₂F₅, n-C₃F₇, i-C₃F₇,
R² is H, halogen, cyano, alkoxycarbonyl, hydroxymethyl, haloalkyl, alkylthioalkyl, alkoxyalkyl, acyloxyalkyl, formyl, thiocyanatoalkyl, alkylsulfonyl, alkylthio, alkoxyiminomethyl, benzyloxyiminomethyl being optionally substituted by one or more of halogen, alkyl, alkoxy, cyano, nitro, vinyl being optionally substituted by one or more halogen, nitro or cyano, styryl being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy, or phenyl being optionally substituted by one or more of halogen, cyano, alkyl, alkenyl, alkynyl, haloalkyl, alkoxy, nitro, alkoxycarbonyl, alkylcarbonyloxy, alkylenedioxy, alkylcarbonyl, amino, alkylamino, haloalkoxy, alkylthio, alkylsulfonyl, haloalkenyl, alkoxycarbonylalkyl or alkoxycarbonylalkoxy; thiocyanatoalkyl,
R³ is H, alkyl, alkoxyalkyl or alkoxy,
R⁴ is aryl (especially phenyl, naphthyl, pyridinyl, pyrimidinyl, thienyl, furyl, thiazolyl, isothiazolyl, pyrazolyl, oxazolyl, isoxazolyl, thiadiazolyl (1,2,4, and 1,3,4), oxadiazolyl (1,2,4- and 1,3,4); being optionally substituted by one or more of halogen, cyano, alkyl, haloalkyl, alkoxy, nitro, alkoxycarbonyl, alkylcarbonyloxy, alkylcarbonyl, amino, alkylamino, haloalkoxy, alkylthio, or alkylsulfonyl, haloalkylsulfonyl), X is O, S or NR⁵, and
R⁵ is alkyl, cycloalkyl, alkoxy, alkenylalkyloxy or alkynylalkyloxy.
